(19) United States
(12) Patent Application Publication
(10) Pub. No.: US 2011/0030256 A1
(43) Pub. Date: Feb. 10, 2011

(54) Adjustable and reusable identification bracelet with concealed compartment and secure locking mechanism

(76) Inventor: Ginger W. Juliano, Westport, CT (US)

Correspondence Address: Ginger W. Juliano, 14 Kirock Place, Westport, CT, 06880, US

(21) Appl. No.: 12/798,393

(22) Filed: Apr. 2, 2010

Related U.S. Application Data
(60) Provisional application No. 61/211,929, filed on Apr. 6, 2009.

(51) Int. Cl. A44C 5/00; A61B 5/117

(52) U.S. Cl. 40/633

(57) ABSTRACT

Adjustable and Reusable Identification Bracelet with Concealed Compartment and Secure Locking Mechanism for identifying individuals. The device comprises one individual strap of polypropylene webbing which can fasten securely around an individual's wrist. The opposite ends of the strap lock together to form a continuous identification bracelet around an individual's wrist. An adjustment means is incorporated onto the strap to lengthen or shorten the bracelet to ensure a proper fit. The bracelet further comprises a concealed fabric compartment beneath the bracelet to secure identification information on a plastic card. Accordingly, the device may be used as a means of identification and identification storage.

### CROSS REFERENCE TO RELATED APPLICATIONS

The present application derives priority from U.S. Provisional Patent Application No. 61/211,929 for “IDENTIFICATION BRACELET WITH SECURE LOCKING MECHANISM”; filed on Apr. 6, 2009.

### STATEMENT REGARDING FEDERALLY SPONSORED RESEARCH OR DEVELOPMENT

“NOT APPLICABLE”

### REFERENCE TO SEQUENCE LISTING, A TABLE, OR A COMPUTER PROGRAM

### LISTING COMPACT DISC APPENDIX

“NOT APPLICABLE”

### BACKGROUND OF THE INVENTION

1. Field

The invention relates to identification bracelets, and is concerned in particular with the provision of an improved wristband, anklet or bracelet carrying identification information.

2. Background of the Invention

Conventional identification bracelets are made of either metal which a security alarm can trigger off and are typically intended for medical information. Other types of identification bracelets similar to the hospital style are frequently made of a non-reusable type material that once locked into position need to be cut with scissors to be removed. This is inconvenient from an economical and environmental stand point if a person would like to have a bracelet that they used over and over again to identify themselves. Other identification bracelets exist such as the velcro style that is made of an adjustable type stretchy material but the locking mechanisms are not very secure. These stretchy, velcro style bracelets are not secure and can easily be removed by a child. Once the velcro is merely tugged on by a child, the bracelets lose their intended purpose of serving as a means of identification. As many as 150,000 children are reported missing annually.

To protect the safety of individuals when they are unable to speak for themselves in emergency situations, it is appropriate to have a convenient method for first responders to identify the individual with an identification bracelet. Many individuals such as the elderly, young children and athletes do not carry identification information in an easily visible location. Precious time can be lost searching for the appropriate identification.

Absent such conveniently located identification information on a bracelet which is readily visible and available for first responders to use, first responders will be delayed in their ability to provide necessary assistance in emergency situations and to contact family members. Such dangers are present in situations of travel or athletic individuals who are injured or elderly individuals and young children who wander off without identification and are unable to effectively communicate their care giver's contact information or their identification information.

The invention is a wrist or ankle band which holds items of identification on the person or animal. The device will identify the person or animal. The invention has a secure locking mechanism. The invention contains a concealed compartment in which the individual's contact information can be removed to identify the individual. The device will help reunite lost individuals, children, adults or pets with their loved ones. The device contains identification information in a hidden compartment located inside the bracelet, band or anklet. The device is also adjustable.

How does the bracelet work? If your loved one, adult, child or pet wanders off or gets injured and does not remember your name, address, or telephone number or is unable to communicate; a secret pocket in the bracelet contains an information card where parents or loved ones can write their contact information. Caring individuals could then remove the identification card and contact the parents or loved ones to ensure the individual's safe return home or information provided for medical purposes could be revealed. The comfortable, adjustable, reusable safety identification bracelet with secure locking mechanism has been developed for individuals to wear while traveling, shopping, at playgrounds, amusement parks, hospitals or convalescent homes.

The device is to be worn on the wrist or ankle and is adjustable to fit most wrist or ankle sizes. The bracelet, band or anklet can be worn by individuals while exercising, elderly people, young children or pets or anyone who would like to be identified if in the event that they are unable to speak for themselves. The bracelet device or band has a unique and secure locking mechanism. There are two clips that slide together and then there is a button that enables the clips to be locked. There is no metal in the bracelet so when individuals are traveling through security at airports the alarms will not go off and children, as well as the elderly and adults can have their identification in a concealed yet readily accessible device.

What led me to the invention was the fact that one day I was out jogging with my baby in a jogging stroller carriage and I was going up a hill on a sunny day and the cars were going by really fast and I got scared that neither my baby nor I had any identification on us. If I got hurt or the baby, nobody would know who we were or who to contact if in the event we were unable to speak for ourselves. I also thought of the fact later on that lots of kids wander off from their parents and they don't know their contact information or are not able to speak yet and they need to be properly identified if they are to be reunited with their families in a timely manner.

Identification bracelets would make it easier for first responders to be able to identify care givers for children, adults or even the elderly. If the identification bracelets were comfortable, stylish, adjustable, reusable with concealed compartments and the bracelets had secure locking mechanisms, then the children, adults or elderly would be more likely to wear them. The bracelets would not be easily removed and they would actually remain on their wrists if the need required them to be there for identification purposes.

The problem solved by this invention is an identification bracelet made of a comfortable material, nylon polypropylene webbing having an adjustable plastic slider, a secret storage compartment for identification items located in the bracelet in a fabric compartment with the bracelet having a unique locking mechanism to securely fasten the bracelet to the wearer's wrist.

Inventors have created several types of identification bracelets. Publication No.: US 2005/0010993 A1 from Mouritzen, discloses an identification bracelet for kids comprised of polyurethane with a locking construction with round holes and triangles with barb shapes to lock the bracelet in place but if it is pulled on the bracelet will open under tension. This device mentioned is uncomfortable to wear and is not secure. The bracelet mentioned from Mouritzen has a different locking mechanism than the invention that I am presenting. The identification bracelet presented by Mouritzen has the adjustable feature and locking mechanism integrated into one strap of polyurethane. The bracelet that I am presenting has a separate strap, adjustable element and locking mechanism as well as a concealed compartment located on the bottom of the strap in a fabric compartment for storage.

U.S. Pat. No. 5,092,067 to Prout teaches a leather identification bracelet with a metallic identification plate. While leather is flexible, it is expensive and the metallic identification plate is also expensive and may not be convenient for traveling through security alarms at airports. The identification bracelet taught by Prout indicates a securing means comprising tubular studs and screws to attach the identification information in the bracelet. The invention that I am presenting has removable identifying information stored in a concealed compartment made of fabric sewn to polypropylene webbing that is secured in place not by studs or screws but by an adjustable sliding mechanism which makes it easier for a first responder to access the information in a timely manner and with greater ease.

U.S. Pat. No. 7,515,053 to Klein shows an identification band having at least one surface of the band being transparent and the information tag being visible through the transparent surface of the band. The information for identification in my invention is not visible through any transparent surfaces. The information is concealed in a fabric compartment located on the underside of the bracelet and is secured in place by a sliding adjustment where it is not easily visible to keep the information discrete. Furthermore the information on my bracelet is provided on a water proof removable card made of plastic that is able to be written on with either pencil or pen and is capable of being printed on with a computer printer also. Additionally, the locking mechanism in the bracelet described by Klein is different than the locking mechanism in my bracelet. Also, the identification band discussed by Klein is not adjustable which could mean that it may not fit various wrist sizes and as such could fall off the wearer's wrist and lose its ability to serve its intended purpose as a form of identification transport.

### BRIEF SUMMARY OF THE INVENTION

The general idea of the claimed invention(s) is an adjustable identification device to be worn on a body part such as the wrist or ankle of an individual or an animal. The bracelet device solves the previously existing problem by providing an adjustable, reusable, comfortable and convenient means of identification with a concealed compartment. The concealed compartment has a secure locking mechanism feature which makes it more difficult for children to remove than the velcro type identification bracelets currently on the market. Additionally, it is reusable which is an advantage over the hospital type identification bracelets that have to be cut off with scissors to be removed.

The object of the invention is an adjustable feature on an identification bracelet which also contains a concealed identification storage compartment and removable identification card which can be used to discreetly reveal detailed personal contact information about an animal, child or an adult that is either elderly, injured or unable to communicate. Another object of the reusable identification bracelet is the unique and secure locking mechanism. Additionally, this locking mechanism is advantageous over standard velcro style bracelets. The identification bracelet is difficult for children to disengage due to the sliding lock mechanism, yet convenient for parents to remove from the child's wrist or ankle.

It is understood that other embodiments of the present invention will become readily apparent to those skilled in the art from the following detailed description, wherein it is shown and described only exemplary embodiments of the invention by way of illustration. As will be realized, the invention is capable of other and different embodiments and its several details are capable of modification in various other respects, all without departing from the spirit and scope of the present invention. Accordingly, the drawings and detailed description are to be regarded as illustrative in nature and not as restrictive.

### BRIEF DESCRIPTION OF THE SEVERAL VIEWS OF THE DRAWING

Other objects, features, and advantages of the present invention will become apparent from the following detailed description of the preferred embodiments and certain modifications there of when taken together with the accompanying drawings in which:

FIG. 1 is a top view of the assembled identification bracelet

FIG. 2 is a bottom view of the assembled identification bracelet showing the identification tag and concealed compartment FIG. 3 is a view of the male clasp

FIG. 4 is a view of the locking mechanism prior to engagement in the locking position

FIG. 5 is a side view of the adjustable feature sliding mechanism and strap loop

### DETAILED DESCRIPTION OF THE INVENTION

In general, the present invention relates to aids used by individuals to assist them in identification. The invention relates to a novel, lightweight, comfortable, portable, adjustable, easy to operate device made of a strong webbing or fabric type material and plastic. Within the scope and spirit of the contemplated design goals, many different suitable flexible materials, temporary and permanent attachment mechanisms, suitable structure Alternative etc. may be incorporated.

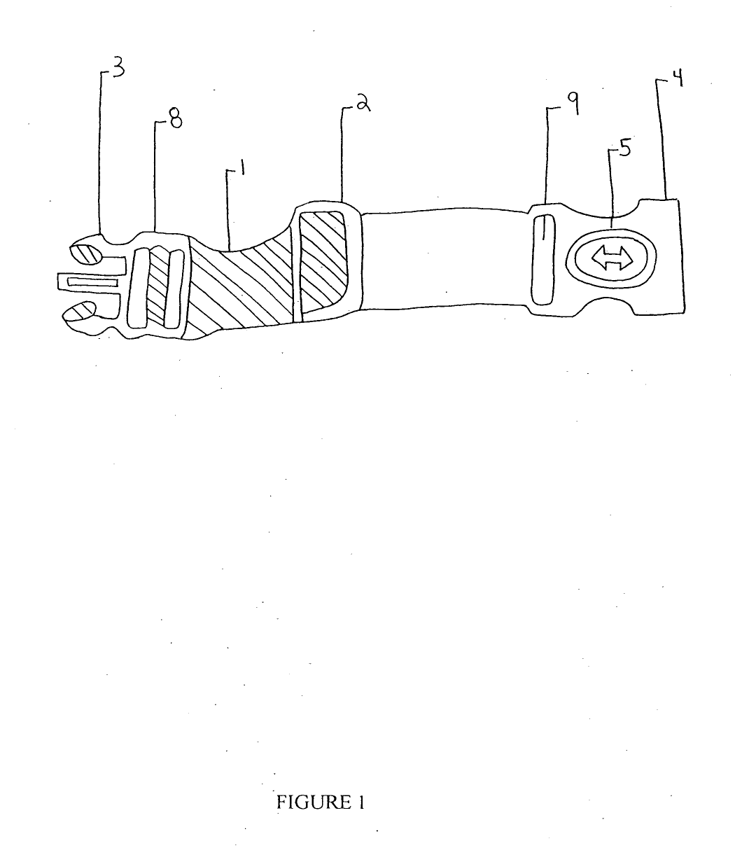

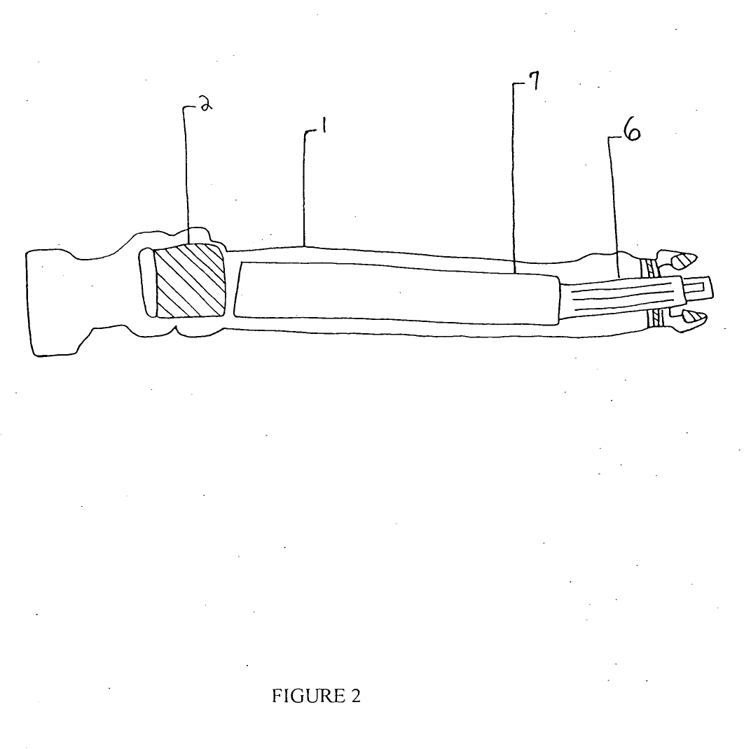

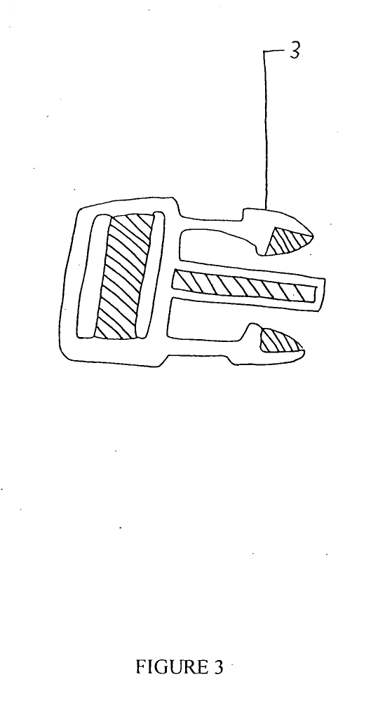

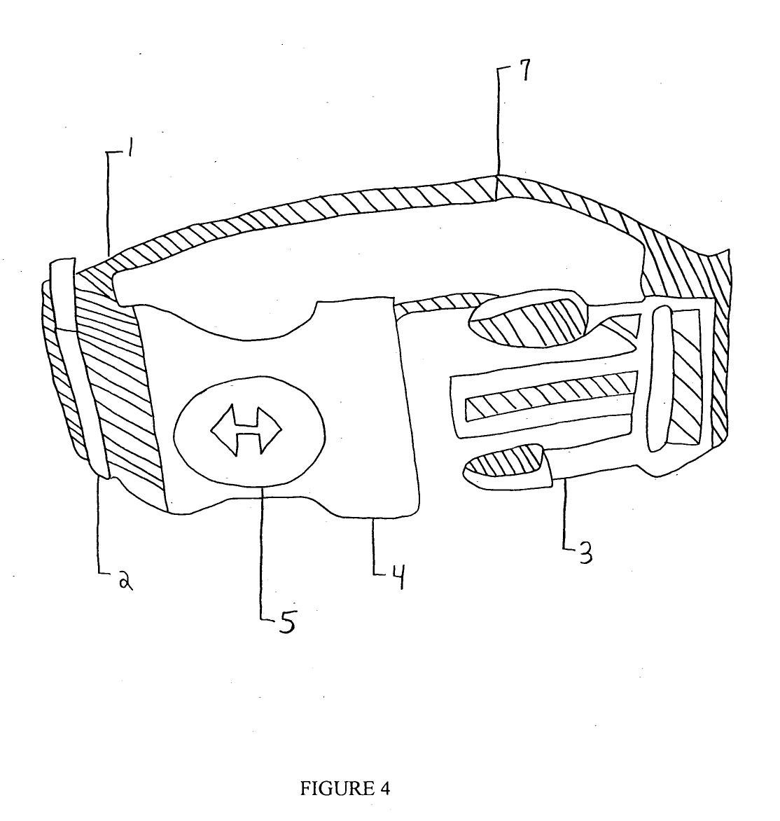

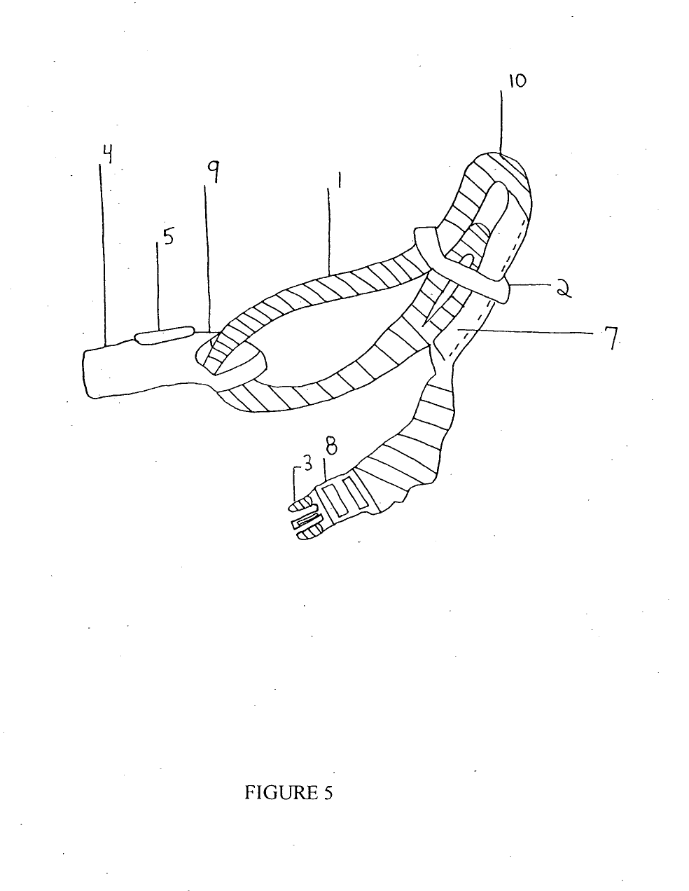

An improved safety identification bracelet is shown in FIG. 1. The identification device generally comprises a single strap portion made of polypropylene webbing 1. The bracelet may be releasable joined together by an interlocking fastener to form a continuous safety identification bracelet. The interlocking fastener is attached to each end of the single strap portion and an adjustable feature sliding mechanism is also located on the strap.
In FIG. 1 single strap is made of polypropylene webbing and is attached by stitching the webbing together after it loops through aperture slot 8. The female connector 4 comprises receptacle for engaging male connector. The male interlocking fastener clasp 3 in FIG. 3 functions to connect with the female interlocking fastener clasp 4 in FIG. 4 by entering a slot located in the female clasp to form a continuous bracelet or anklet. In FIG. 4 the device can be securely locked by sliding locking mechanism 5 to the right which is located on the female clasp connector 4. The male clasp interlocking fastener 3 in FIG. 4 can be disengaged to open the bracelet by pushing to the left the sliding lock 5 which is located on the female clasp 4.
Attachment mechanism 3 and 4 in FIG. 1 comprises plastic, but may alternatively comprise metal or any other durable material.
As shown in FIG. 4 end 3 engages attachment means of female interlocking clasp fastener by passing through a central opening and sliding locking mechanism.
The end of the strap is of a polypropylene webbing construction which is flexible, durable and comfortable. Alternatively, strap is a suitable fabric material, leather or plastic type material.
Webbing of present invention is a polypropylene strap inserted through the web receiving aperture of the slide mechanism which functions as an adjusting element enabling the bracelet to be adjustable in size allowing the user to reduce or increase the length of the identification bracelet.
Polypropylene webbing strap is then inserted through the web receiving aperture of the female clasp with the sliding lock mechanism.
The polypropylene webbing strap is then inserted through the adjusting element and then loops around the web receiving aperture of the male portion of the clasp.
FIG. 2 Shows the bracelet contains a hidden compartment 7 for storage of identifying information on a plastic card which is inserted into the concealed compartment 7 located on the underside of the bracelet for confidentiality of the wearer's information yet for ease of access in emergency situations the identify of the wearer can be accessed.
In FIG. 2, the identifying information of the wearer is located on the plastic identification card 6 which is revealed by sliding back the sliding the sliding adjustable mechanism 2 on the polypropylene webbing strapl and pulling out the identification card 6.
In FIG. 2 the identification can be reinserted into the hidden storage compartment 7. The adjusting element 2 functions to reduce or enlarge the size of the bracelet and to help secure the identifying information by sliding back into place over the hidden identification storage compartment.
The reusable identification bracelet allows the user to customize the fit by an adjusting element with adjustable webbing which enables the bracelet to be reduced or enlarged in length. There is a fastening means associated with said strap for detachably fastening strap to a wearer's wrist. In FIG. 1 polypropylene webbing strap 1 carries an adjustment means 2 which is preferably a slide fastener of the type well known in the prior art and within the public domain. Adjustment means 2 preferably comprises a rectangular fastener having a length slightly longer than the width of strap 1. Adjustment means extends through the length of the strap from side to side. Once through slot 9, end of strap 1 reverses direction and continues along to rear face of the strap 1 toward an engaging and adjustment means 2 and finally ending in a loop configuration 10 on FIG. 5.
The strap is easily shortened and lengthened by threading strap 1 through adjustment means sliding mechanism 2 from one side and pulling from the other side to take up the slack. The foregoing description with attached drawings is only illustrative of possible embodiments of the described method and should only be construed as such. Other persons of ordinary skill in the art will realize that many other specific embodiments are possible that fall within the scope and spirit of the present idea. The scope of the invention is indicated by the following claims rather than by the foregoing description. Any and all modifications which come within the meaning and range of equivalency of the following claims are to be considered within their scope.

The invention claimed is:

1. Identification bracelet made of polypropylene webbing or flexible material with an adjustable sliding mechanism made of plastic enabling the size of the bracelet to be reduced or enlarged so it is adjustable and better able to fit various wrist sizes. Adjustable sliding mechanism also serves to secure the identification in place in concealed storage compartment.

2. Identification bracelet comprising of a sliding lock mechanism made of plastic which functions as a closure device and enables the identification bracelet or anklet to be attached or removed manually from the body part of the wearer.

3. Identification bracelet with concealed compartment made of polypropylene webbing sewn to fabric for identification storage made of fabric beneath identification bracelet with a slide in and out, removable water proof identification component made of plastic. Adjustable sliding mechanism also serves to secure the identification in place in concealed storage compartment.

4. The fabric concealed compartment of claim 3 is not transparent.

5. Identification bracelet with concealed compartment of claim 3 wherein the information tag is not viewable from the bracelet unless it is physically removed.

6. Identification bracelet with concealed compartment of claim 3 wherein it is sealed shut with the same adjustable sliding mechanism that also enables the bracelet to be enlarged or reduced in size.

7. Identification bracelet with concealed compartment of claim 3 wherein the identification information can be accessed by first responders or caring individuals by sliding back manually the movable lock located on the female clasp and then sliding back the adjustable sliding element and manually pulling out the information card to reveal the contact or medical information which is discreetly located.
